(19) United States
(12) Patent Application Publication
(10) Pub. No.: US 2014/0276401 A1
(43) Pub. Date: Sep. 18, 2014

(54) Catheter Shaft and Method of Forming Same

(71) Applicant: ABBOTT CARDIOVASCULAR SYSTEMS INC., SANTA CLARA, CA (US)

(72) Inventors: Jeong S. Lee, Diamond Bar, CA (US); Kenneth L. Wantink, Temecula, CA (US)

(73) Assignee: ABBOTT CARDIOVASCULAR SYSTEMS INC., SANTA CLARA, CA (US)

(21) Appl. No.: 14/212,966

(22) Filed: Mar. 14, 2014

Related U.S. Application Data
(60) Provisional application No. 61/787,415, filed on Mar. 15, 2013.

(51) Int. Cl. A61M 25/00; A61M 25/10

(52) U.S. Cl. CPC A61M 25/0045; A61M 25/10; A61M 25/0009
USPC 604/96.01; 604/523; 156/244.13

(57) ABSTRACT

Elongate, flexible catheter includes an elongated shaft having a proximal end, a distal end, and a lumen defined therein. The shaft includes a tubular member having an outer layer and an inner layer. The outer layer includes a first polymer selected from the group consisting of nylon 12, polyether block amide, and combinations thereof. The inner layer includes a second polymer having a heat deflection temperature greater than about 53° C. selected from the group consisting of nylon 11, nylon 6, nylon 6,6, nylon 6,12, polyamide-imide, polyetherimide, polypropylene, polyethylene terephthalate, polybutylene terephthalate, polyethereetherketone, and combinations thereof. Method of making an elongate, flexible catheter is also provided.

### CROSS-REFERENCE TO RELATED APPLICATION

This application claims priority to U.S. Provisional Patent Application Ser. No. 61/787,415, entitled “Multilayer Balloon for a Catheter,” filed Mar. 15, 2013, the contents of which are fully incorporated herein by reference.

### BACKGROUND

1. Field

The presently disclosed subject matter relates to intraluminal catheters for use in percutaneous transluminal coronary angioplasty (PTCA) or stent delivery systems or the like. Particularly, the disclosed subject matter relates to an improved catheter shaft that provides pushability when exposed to body temperature.

2. Description of Related Art

Intraluminal catheters are well known and beneficial for a variety of medical uses, including diagnostics, therapeutics, and treatment. For example, and not limitation, balloon catheters can be used for a number of different vascular and/or coronary applications. In percutaneous transluminal coronary angioplasty (PTCA) procedures, a guidewire is typically advanced into the coronary artery until the distal end of the guidewire crosses a lesion to be dilated. A dilatation catheter having an inflatable balloon on the distal portion thereof is advanced into the coronary anatomy over the guidewire until the balloon of the dilatation catheter is properly positioned across the lesion. Once properly positioned, the dilatation balloon is inflated with inflation fluid one or more times to a predetermined size to open up the vascular passageway. Generally, the inflated diameter of the balloon is approximately the same diameter as the native diameter of the body lumen being dilated so as to complete the dilatation, but not over-expand the artery wall. After the balloon is finally deflated, blood flow resumes through the dilated artery and the dilatation catheter and the guidewire can be removed therefrom.

In addition to or as an alternative of angioplasty procedures, it may be desirable to implant an intravascular prosthesis, generally called a stent, inside the artery at the site of the lesion. Stents can also be used to repair vessels having an intimal flap or dissection or to generally strengthen a weakened section of a vessel or to maintain its patency. Stents are usually delivered to a desired location within a coronary artery in a contracted condition on a balloon of a catheter, which is similar or identical in many respects to a balloon angioplasty catheter. The balloon, and thus the stent, is expanded within the patient's artery to a larger diameter. The balloon is deflated to remove the catheter with the stent implanted at the site of the dilated lesion. See for example, U.S. Pat. No. 5,507,768 to Lau et al. and U.S. Pat. No. 5,458,615 to Klemm et al., each of which is hereby incorporated by reference in its entirety. Alternatively, the stent can be delivered to a desired location within a coronary artery in a contracted condition under a retractable sheath of a catheter, which when pulled back allows the stent to expand within the patient's artery to a larger diameter. See for example, U.S. Pat. Nos. 5,360,401, 7,850,724, and 8,257,420 and U.S. Patent Publication Nos. 2013/0304179, 2013/0304181, and 2012/0065644, each of which is hereby incorporated by reference in its entirety.

It is desirable to provide an intraluminal catheter with a shaft that provides pushability when exposed to body temperature for an extended period, e.g., during advancement within the tortuous anatomy of a patient's vascular and performance of PTCA procedures. One challenge with catheter shafts formed of conventional materials (e.g., certain nylons or PEBAX) has been a loss of stiffness after extended exposure in the body, which can cause a loss in pushability because the shaft is not able to transmit proximal force well distally. Accordingly, there remains a need to provide a catheter shaft providing pushability when exposed to body temperature for an extend period while also being easily bondable to the other catheter components (e.g., balloon).

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter provides an elongate, flexible catheter including an elongated shaft having a proximal end, a distal end, and a lumen defined therein. The shaft includes a tubular member having an outer layer including a first polymer selected from the group consisting of nylon 12, polyether block amide, and combinations thereof and an inner layer including a second polymer having a heat deflection temperature greater than about 53° C. selected from the group consisting of nylon 11, nylon 6, nylon 6,6, nylon 6,12, polyamide-imide, polyetherimide, polypropylene, polyethylene terephthalate, polybutylene terephthalate, polyethereetherketone, and combinations thereof. The inner layer and the outer layer can be coextruded. The tubular member can form one or more of a proximal shaft portion, a mid shaft portion, a distal shaft portion, or a sleeve disposed over a working device.

Various suitable materials can be used for the layers of the tubular member. For example, the first polymer can include nylon 12 or PEBAX. The second polymer can be directly bondable to the first polymer and can be selected from the group consisting of nylon 11, nylon 6, nylon 6,6, nylon 6,12, polyamide-imide, polyetherimide, and combinations thereof. Alternatively, the second polymer can be selected from the group consisting of polypropylene, polyethylene terephthalate, polybutylene terephthalate, polyethereetherketone, and combinations thereof, and the tubular member can further include an intermediate layer disposed between the first layer and the second layer. The intermediate layer can include a tie material directly bondable to the first and second polymers, such as ethylene acrylic acid copolymer, ethylene methacrylic acid copolymer, or combinations thereof. The inner layer, the intermediate layer, and the outer layer can be coextruded.

Additionally, the elongate, flexible catheter can include a working device disposed proximate the distal end of the elongated shaft. The working device can include an inflatable balloon.

As embodied herein, the catheter can have improved pushability when exposed to body temperature as compared to a similar catheter having a shaft including a tubular member consisting of the first polymer.

A method of making an elongate, flexible catheter is also provided. The method includes coextruding a tubular member having an outer layer and an inner layer to form at least a portion of an elongated shaft having a proximal end, a distal end, and a lumen defined therein. The outer layer includes a first polymer selected from the group consisting of nylon 12, polyether block amide, and combinations thereof, and the inner layer includes a second polymer having a heat deflection temperature greater than about 53° C. The method also includes disposing a working device proximate the distal section of the elongated shaft. The method of making and the resulting catheter can include any of the features described herein above for the elongate, flexible catheter.

It is to be understood that both the foregoing general description and the following detailed description are exemplary and are intended to provide further explanation of the disclosed subject matter.

The accompanying drawings, which are incorporated in and constitute part of this specification, are included to illustrate and provide further understanding of the disclosed subject matter. It will be appreciated that the drawings are not to scale, and are provided for purposes of illustration only. Together with the description, the drawings serve to explain the principles of the disclosed subject matter.

### BRIEF DESCRIPTION OF THE DRAWINGS

FIG. 1 schematically depicts a representative embodiment of a catheter in accordance with certain aspects of the disclosed subject matter.

FIG. 2 is an enlarged view of the circular section labeled FIG. 2 in FIG. 1.

FIG. 3 is a transverse cross-sectional view of an embodiment of the catheter shaft along line 3-3.

FIG. 4 is a transverse cross-sectional view of an alternative embodiment of the catheter shaft along line 3-3.

FIG. 5 is a graph of tan delta vs. temperature for exemplary PEBAX materials.

FIG. 6 schematically depicts another representative embodiment of a catheter in accordance with certain aspects of the disclosed subject matter.

FIG. 7 is a transverse cross-sectional view of an embodiment of the catheter shaft along line 7-7.

FIG. 8 is a transverse cross-sectional view of an alternative embodiment of the catheter shaft along line 7-7.

### DETAILED DESCRIPTION

The devices and methods presented herein can be used for a variety of treatments within various lumens of a patient. For example, the disclosed subject matter is suited for treatment of the cardiovascular system of a patient, such as performance of angioplasty and delivery of a therapeutic agent and/or a stent to a vasculature. In accordance with the disclosed subject matter, an elongate, flexible catheter is provided including an elongated shaft having a proximal end, a distal end, and a lumen defined therein. The shaft includes a tubular member having an outer layer comprising a first polymer selected from the group consisting of nylon 12, polyether block amide, and combinations thereof and an inner layer comprising a second polymer having a heat deflection temperature greater than about 53° C. selected from the group consisting of nylon 11, nylon 6, nylon 6,6, nylon 6,12, polyamide-imide, polyetherimide, polypropylene, polyethylene terephthalate, polybutylene terephthalate, polyethereetherketone, and combinations thereof.

Reference will now be made in detail to the preferred embodiments of the disclosed subject matter, examples of which are illustrated in the accompanying drawings.

For the purpose of illustration and not limitation, FIGS. 1-3 illustrate an over-the-wire type balloon catheter 10 in accordance with the disclosed subject matter. Catheter 10 includes an elongated catheter shaft 11 having a proximal end, a distal end, and a lumen (e.g., 18 in FIG. 3) defined therein. The shaft 11 includes a proximal shaft section 12, a distal shaft section 13, an outer tubular member 14, and an inner tubular member 15. Inner tubular member 15 defines a guidewire lumen 16 adapted to slidingly receive a guidewire 17, and the coaxial relationship between outer tubular member 14 and inner tubular member 15 defines annular inflation lumen 18 (see FIG. 3, illustrating transverse cross sections of the catheter 10 of FIG. 1, taken along line 3-3). An inflatable balloon 19 is disposed on the distal shaft section 13, having a proximal skirt section 30 sealingly secured proximate the distal end of outer tubular member 14, and a distal skirt section 31 sealingly secured proximate the distal end of inner tubular member 15, so that its interior is in fluid communication with inflation lumen 18.

An adapter 20 at the proximal end of the shaft is configured to provide access to guidewire lumen 17, and to direct inflation fluid through arm 21 into inflation lumen 18. Balloon 19 has an inflatable working length 32 located between proximal tapered cone section 33 and distal tapered cone section 34 of the balloon. FIG. 1 illustrates the balloon 19 in an noninflated configuration prior to inflation. The distal end of catheter can be advanced to a desired region of a patient's body lumen in a conventional manner, and balloon 19 inflated to perform a procedure such as dilatation of a stenosis.

In the embodiment illustrated in FIGS. 1-3, the outer tubular member has a proximal section 25, and a distal section 26. As best illustrated in FIG. 2, showing an enlarged longitudinal cross sectional view of the section of the catheter 10 shown in FIG. 1, taken within circle 2, the proximal section 25 is multilayered with a first inner layer 27 and a second outer layer 28. The outer layer 28 can be any suitable polymer having a heat deflection temperature less than about 45° C. For example, the outer layer 28 can comprise a polymer selected from the group consisting of nylon 12, polyether block amide, and combinations thereof. Additionally or alternatively, outer layer 28 can comprise polyurethane. The second inner layer 27 can comprise a second polymer having a heat deflection temperature greater than about 53° C. For example, the inner layer 27 can comprise a polymer having a heat deflection temperature greater than about 53° C. selected from the group consisting of nylon 11, nylon 6, nylon 6,6, nylon 6,12, polyamide-imide, polyetherimide, polypropylene, polyethylene terephthalate, polybutylene terephthalate, polyethereetherketone, and combinations thereof. The multilayer tubular member 25 can be formed by coextruding a tubular product formed from the two polymeric components to create a tubular member having an outer layer 28 and an inner layer 27 of the two polymeric materials using a coextruder, as known to one of ordinary skill in the art.

Catheter shafts including one or more multilayer tubular members in accordance with the disclosed subject matter can provide improved pushability when exposed to body temperature for an extended period, e.g., during advancement within the tortuous anatomy of a patient's vascular and performance of PTCA procedures, as compared to a similar catheter having a shaft including a tubular member consisting of the first polymer. Indeed, catheter shafts formed only of conventional materials including certain nylons or PEBAX can lose stiffness after extended exposure in the body, which can cause a loss in pushability because the shaft is not able to transmit proximal force well distally. This loss of stiffness can result if the glass transition temperature or heat deflection temperature of the shaft material is close to or less than that of body temperature (i.e., 37° C.). For example, certain suitable grades of polyether block amide, commercially available as PEBAX from Arkema (e.g., having a Shore durometer hardness of 63D, 70D, and 72D) have a glass transition temperature near or below body temperature as can been seen at the peak in the tan delta vs. temperature graph shown in FIG. 5. Similarly, certain grades of nylon can have a glass transition temperature and/or heat deflection temperature near body temperature. For example, Rilsan PA 12 has a glass transition temperatures of 35° C., and EMS L25 nylon 12 has a heat deflection temperature of 45° C. at 1.82 MPa.

The heat deflection temperature (“HDT”) of a material is closely related to the glass transition temperature and can be determined experimentally as disclosed in ASTM D648 by loading a test specimen in three-point bending in the edgewise direction. The stress used for testing can be 0.455 MPa or 1.82 MPa, and the temperature is increased at 2° C./min until the specimen deflects 0.25 mm. As such, the heat deflection temperature measures the temperature a material starts to lose stiffness when under load. As such, the heat deflection temperature is relevant to pushing or advancing a catheter shaft through the vascular system of patient and can be a better indicator than the glass transition temperature for transmission of proximal force as the tubular member is normally under proximal load during an intervention. Heat deflection temperature should therefore be lower than the glass transition temperature of a material because of the applied load.

Therefore, a catheter shaft formed of a polymer (e.g., certain grades of nylon or PEBAX) having a heat deflection temperature near body temperature can lose stiffness and pushability when exposed to body temperature for an extended period. By contrast, the multilayer tubular member in accordance with the disclosed subject matter includes an inner layer of second polymer having a heat deflection temperature greater than about 53° C., which will reduce the loss of stiffness when exposed to body temperature for an extended period. As such, the catheter shaft can remain pushable during advancement of the catheter within the tortuous anatomy of a patient's vascular.

Exemplary second polymers for inner layer 27 having a heat deflection temperature greater than about 53° C. include, but are not limited to, nylon 11 (HDT at 1.82 MPa of 82° C.), nylon 6 (HDT at 1.82 MPa of 65-80° C.), nylon 6,6 (HDT at 1.82 MPa of 100° C.), nylon 6,12 (HDT at 1.82 MPa of 65° C.), polyimide (HDT at 1.82 MPa of 360° C.), polyamide-imide (HDT at 1.82 MPa of 279° C.), polyetherimide (HDT at 1.82 MPa of 190° C.), polypropylene (HDT at 1.82 MPa of 65° C.), polyethylene terephthalate (HDT at 1.82 MPa of 80° C.), polybutylene terephthalate (HDT at 1.82 MPa of 60° C.), polyethereetherketone (HDT at 1.82 MPa of 160° C.), and combinations thereof

In some embodiments of the disclosed subject matter, and as illustrated in FIGS. 1-3, the second layer 28 is in direct contact with the first layer 27 around a circumference of the first layer 27. Thus, the second layer 28 is not separated from the first layer 27 by an intermediate layer or braid. Further, the second layer 28 can be a solid-walled layer, which is not itself a braid or mesh. In embodiments wherein the second layer 28 is in direct contact with the first layer 27 around a circumference of the first layer 27, the second layer 28 is preferably made of a second polymer that is directly bondable to the first polymer of the first layer 27. When the first polymer comprises nylon or PEBAX, suitable second polymers that are directly bondable to the first polymer include but are not limited to nylon 11, nylon 6, nylon 6,6, nylon 6,12, polyimide, polyamide-imide, polyetherimide, and combinations thereof.

Alternatively, in some embodiments of the disclosed subject matter and as shown in FIG. 4, the tubular member includes an intermediate layer 40 disposed between the first layer 27 and the second layer 28. The intermediate layer can provide a number of benefits including but not limited to an improved moisture barrier, improved binding to the materials of the first layer and the second layer and reduced delamination during further processing of the catheter shaft (e.g., thermal bonding to other catheter components).

For example, the intermediate layer 40 can improve the binding of non-compatible or less-compatible first and second polymers and reduce delamination thereof during further processing of the catheter shaft including but not limited to thermal bonding to other catheter components (e.g., balloon and/or other shaft sections). In some embodiments, when the first polymer comprises nylon or PEBAX and the second polymer comprises polypropylene, polyethylene terephthalate, polybutylene terephthalate, polyethereetherketone, and combinations thereof, an intermediate tie layer can be provided that is directly bondable to the first and second polymers. The intermediate layer can include any suitable tie material known to one of ordinary skill in the art such as ethylene acrylic acid copolymers, available commercially as Primacor EAA from Dow Chemical, ethylene methacrylic acid copolymerss, available commercially as Nucrel from DuPont, and/or Plexar tie layer resin available from LyondellBasell.

In some embodiments, certain nylons suitable for the inner layer 27 can be sensitive to moisture, for example in the vascular environment of the body. It is noted that each of nylon 6, nylon 6,6, and nylon 6,12 is a hygroscopic material, i.e., sensitive to moisture, wherein the absorption of moisture can reduce tensile strength and flexural modulus of the material. As such, and as embodied herein, an outer layer 28 can be provided that less hydroscopic than the inner layer 27, which can protect the moisture sensitive layer. The outer layer 27 can, for example, comprise a low hygroscopic polymer, i.e., less sensitive to moisture than the intermediate layer. Additionally, the outer layer can be compatible with the inner layer to simplify manufacture and to reduce layer delamination. For example and without limitation, the outer layer 28 can comprise nylon 11, nylon 12, and/or copolymers of nylon 11 or nylon 12, such as a polyether block amide (PEBA) material (e.g., commercially available as PEBAX®).

For the purpose of illustration, Table 3 summarizes the water absorption (per ASTM D570 or ISO 62) of nylon 6, nylon 6,12, and nylon 6,6 at 50% relative humidity and at saturation as compared to the nylon 11, nylon 12, and PEBAX (suitable exemplary polymers for the outer layer).

TABLE 3

| water | nylon | nylon | nylon | nylon 11, i.e., | nylon 12, i.e., | PEBAX | PEBAX | PEBAX |
|---|---|---|---|---|---|---|---|---|
| absorption | 6 | 6,6 | 6,12 | Rilsan PA 11 | Rilsan PA 12 | 72D | 70D | 63D |
| at | 3.3 | 3.2 | 1.5 | 0.9 | 0.8 | 0.7 | 0.7 | 0.7 |
| equilibrium | | | | | | | | |
| 50% RH | | | | | | | | |
| at | 10.5 | 8.5 | 2.8 | 1.9 | 1.8 | 0.9 | 1.1 | 1.1 |
| saturation | | | | | | | | |

As shown in Table 4 below, when exposed to 50% relative humidity, at room temperature, nylon 6,12 demonstrates a reduction of about 38% of tensile modulus, and nylon 6,6 demonstrates a reduction of about 60% of tensile modulus (per ASTM D638 or ISO 527). Similar reductions are observed in flexural modulus (per ASTM D790 or ISO 178) for nylon 6 and nylon 6,6 as shown below in Table 5 below.

TABLE 4

| | |
|---|---|
| Tensile modulus of nylon 6,6 | Tensile modulus of |
| i.e. Zytel E51HSB | nylon 6,12 i.e. Zytel 158 |

| DAM i.e. dry | 50% RH | DAM i.e. dry | 50% RH |
|---|---|---|---|
| 3000 MPa | 1200 MPa | 2400 MPa | 1500 MPa |

TABLE 5

| | | Flexural | |
|---|---|---|---|
| | Flexural | modulus - | % reduction in |
| | modulus - | 50% RH and | flexural |
| | dry and 23° C. | 23° C. | modulus |
| nylon 6 | 2200 MPa | 1200 MPa | 45 |
| nylon 6,6 | 3000 MPa | 1250 MPa | 58 |
| nylon 11 (Rilsan PA 11) | 1200 MPa | 1100 MPa | 8 |
| nylon 12 (Rilsan PA 12) | 1100 MPa | 1000 MPa | 9 |

As demonstrated by the illustrative data above, the construction of a catheter shaft section in accordance with the disclosed subject matter can be used to inhibit or limit reduction in the performance of an inner layer made of a hygroscopic material. For example, a catheter shaft section having an inner layer made of nylon 6, nylon 6,6, or nylon 6,12 protected by outer layer made of nylon 11 or nylon 12 or copolymers thereof, can provide the benefits of the inner layer without a reduction in performance when exposed to moisture. As such, the stiffness, strength, and/or pushability of the catheter component can be maintained. Additionally or alternatively, a thinner catheter shaft section can be provided without sacrificing stiffness, strength, or pushability.

In some embodiments having an inner layer 27 sensitive to moisture, e.g., comprising nylon 6, nylon 6,6, and nylon 6,12, an intermediate layer 40 can be used to provide an improved moisture barrier. For example, tie materials such as Primacor EAA (ethylene acrylic acid copolymer) are less hydroscopic than the inner layer 27, which can further protect the moisture sensitive layer.

Additionally or alternatively, in some embodiments, a multilayer catheter shaft component having an inner layer 27 sensitive to moisture can include an innermost layer (not shown) on the inside of inner layer 27 to further protect or encapsulate the hygroscopic inner layer 27. Suitable materials for the inner most layer include nylons, such as nylon 11, nylon 12, and/or copolymers of nylon 11 or nylon 12, such as a polyether block amide (PEBA) material (e.g., commercially available as PEBAX®).

In the embodiment of FIGS. 1-3, for illustration and not limitation, the second layer 28 of the proximal section 25 forms an outer surface of the multilayered section of the outer tubular member 14, although a coating such as a lubricious coating conventionally used on catheter shafts can optionally be provided on at least a section of an outer surface of the multilayered shaft section. In some embodiments, the first layer 27 forms an inner surface of the multilayered section of the outer tubular member 14. Alternatively, additional layers can be provided.

In the embodiment illustrated in FIG. 1, the distal section 26 of the outer tubular member 14 comprises a single layered tubular member 29, with a proximal end bonded to a distal end of the proximal section 25 of the outer tubular member 14. In a presently preferred embodiment, the distal section 26 is formed of a polymeric material, such as polyether block amide (PEBAX), which is compatible with a polyamide material such as PEBAX and nylon, forming the second layer 28 of the proximal section 25, to allow for fusion bonding the two sections together. However, a variety of suitable methods of bonding can be used including adhesive bonding. Additionally, although a lap joint is illustrated in FIG. 2 between the proximal and distal sections 25/26, a variety of suitable joints can be used including a butt joint, or a lap joint in which the outer diameter of the proximal section 25 is reduced at the joint so that the distal section 26 is flush with the proximal section.

While the embodiment shown in FIG. 1 includes proximal shaft section 25 that is multilayered and a distal section 26 that is a single layer, any portion(s) of the catheter shaft can be multilayered to provide the catheter with the benefits described herein.

For example and in some embodiments, the distal shaft section 26 of the outer tubular member 14 can comprise a multilayered tubular member and include at least an inner layer 27 and an outer layer 28 including any of the materials of construction, features, and/or layers as described herein. In those embodiments, the proximal shaft section 25 can be single layer, multi-layer, or comprise a hypotube as known to one of ordinary skill in the art. Having a multilayer distal section 26 including an outer layer 28 of nylon 12 or PEBAX can provide ease of bonding to the balloon 19, made of typical balloon materials such as nylon and/or PEBAX. For example, the proximal skirt section 30 of the balloon 19 can be fusion bonded to an outer layer 28 of distal shaft section 26, for example, by applying heat to the area of overlap. For example and without limitation, electromagnetic energy, such as thermal, laser, or sonic energy, can be applied to the proximal skirt section 30 of the balloon 19 to bond at least a portion of the proximal skirt section 30 to outer layer 28 of distal shaft section 26. Heating the proximal skirt section of the balloon causes the polymeric material of the balloon 19 to soften, or melt and flow. In some embodiments, a heat shrink tubing (not shown) can be positioned around the outside of proximal skirt section 30 of the balloon 19. The heat shrink tubing, also referred to as a “heat shrink sleeve,” can be composed of a polymeric material configured to shrink when exposed to heat. U.S. Pat. No. 7,951,259, which is hereby incorporated by reference in its entirety, discloses the use of a heat shrink sleeve in fabricating a catheter with a flexible distal end. The heat shrink tubing, when heated, shrinks and exerts an inward radial force on the proximal skirt section 30. With the polymer of the proximal skirt section 30 in a molten or softened, the diameter of the proximal sleeve will be reduced by the force exerted by the heat shrink tubing. After the balloon is cooled, the heat shrink tubing can then be removed. Heating can be accomplished, for example, by laser heating (e.g., using a CO₂ laser), contact heating (e.g., using aluminum nitride, resistance, RF), hot air, resistance heating, induction heating or the like. As embodied herein, for purposes of illustration and not limitation, a solid state laser can be used to heat the shrink tubing and soften the proximal skirt section 30. As a result, the outer surface of the proximal skirt section 30 can be tapered proximally to a smaller outer diameter, while the proximal skirt section 30, in its softened or molten state, can bond to the outer surface of distal shaft section 26. The distal skirt section 31 of balloon 19 can be bonded with the distal section of the inner tubular member 15, in the same manner, which can provide a tapered atraumatic distal end region (or tip) of the catheter.

In an alternative embodiment (not shown), the proximal section 25 and distal section 26 of the outer tubular member can be a single continuous multilayer tubular member 14 that can include any of the features, layers, and/or materials described above for the multilayered proximal section 25. In addition to providing the benefit of improved pushability when exposed to body temperature for an extended period, e.g., during advancement within the tortuous anatomy of a patient's vascular and performance of PTCA procedures, a single piece construction can reduce delamination issues by reducing the number of joints and provide ease of direct bonding to the balloon 19. The single piece shaft can include one or more tapered section(s), which can provide a change in stiffness along the catheter from a more stiff proximal section to a more flexible distal section, as described in detail in U.S. Patent Publication No. 2013/0178795, which is incorporated by reference in its entirety.

In accordance with one aspect of the disclosed subject matter, the relative thicknesses of the layers of a multilayer shaft section can vary depending on the desired properties and function of the shaft section. For example, a catheter shaft section can include first layer 27 having a wall thickness of about 50% of the overall thickness and a second layer 28 having a wall thickness of about 50% of the overall thickness. If a stiffer (and more pushable) shaft section is desired, the relative thickness of the first layer 27 can be increased. For example, in some embodiments the first layer can have a wall thickness greater than 50%, 60%, 70%, or 80% of the overall thickness. By contrast, if a more flexible shaft section is desired, the relative thickness of the first layer 27 can be decreased. For example, in some embodiments, the first layer can have a wall thickness less than 50%, 40%, 30%, or 20% of the overall thickness. In embodiments having an intermediate (e.g., tie) layer, the thickness of the intermediate layer can be about 5% of the overall thickness. For example, the first layer can have a wall thickness of about 45%, the second layer can have a wall thickness of about 50%, and the intermediate layer can have a wall thickness of about 5% of the overall thickness. The thickness of each layer can be tailored to provide the desired properties using any of the thickness as described above.

In accordance with one aspect, the inner tubular member 15 can comprise a single material of monolithic construction or a multi-layered tube. For example, the inner tubular member can be a multilayered tubular member and include at least an inner layer 27 and an outer layer 28 including any of the materials of construction, features, and/or layers as described herein. Additionally or alternatively, the inner tubular member 15 can include a lubricious inner liner and bondable outer layer such as nylon or Pebax, or any of other suitable materials for the intended purpose. In one embodiment, the inner tubular member 15 can include a first inner layer comprising a high density polyethylene (HDPE), a second intermediate layer comprising an adhesive layer, e.g., Primacor, and third outer layer comprising Pebax. Other examples of suitable materials are identified in U.S. Pat. Nos. 6,277,093 and 6,217,547, each of which is hereby incorporated by reference in its entirety. The inner tubular member 15 can be formed by conventional methods including but not limited to extrusion or coextrusion.

For the purpose of illustration and not limitation, FIGS. 6-8 illustrate an alternative embodiment of the disclosed subject matter, in which the balloon catheter 50 is a rapid exchange catheter. As illustrated in FIG. 6, catheter 50 includes an elongated catheter shaft 51 having a proximal end, a distal end, a proximal shaft section 52, a distal shaft section 53, and a lumen 58 defined therein. The elongated shaft 51 includes an outer tubular member 54 and an inner tubular member 55. Inner tubular member 55 has a guidewire lumen 56 defined therein that is adapted to slidingly receive a guidewire 57. Inflation lumen 58 is defined by the outer tubular member 54. An inflatable balloon 59 is disposed on the distal shaft section 53, having a proximal skirt section 70 sealingly secured to the distal end of outer tubular member 54, and a distal skirt section 71 sealingly secured to the distal end of inner tubular member 55, so that its interior is in fluid communication with inflation lumen 58. Balloon 59 also includes a working length 72 between proximal cone section 73 and distal cone section 74. An adapter 60 at the proximal end of the shaft is configured to direct inflation fluid into inflation lumen 58.

In the embodiment illustrated in FIG. 6, the outer tubular member 54 comprises a proximal section 61, a distal section 62, and a midshaft section 63 having a proximal end bonded to the proximal section 61 and a distal end bonded to the distal section 62. A guidewire proximal port 64 in a side wall of the midshaft section 63 is in fluid communication with the lumen 56 of the inner tubular member 55, and with a distal guidewire port in the distal end of the shaft. As shown in FIG. 5, the guidewire 57 exits the catheter proximally from the guidewire proximal port 64 and extends alongside and exteriorly of the proximal section 61 to the proximal end of the catheter 50. Although the guidewire proximal port 64 is in the midshaft section, in an alternative embodiment (not shown) it is located in the proximal section 61 or the distal section 63. Additionally, in an alternative embodiment of rapid exchange catheter 50, the outer tubular member 54 comprises the proximal section 61 directly bonded to the distal section 62, without a midshaft section therebetween (not shown).

As illustrated in FIG. 6 a support mandrel 65 can be disposed in the inflation lumen 58, with a distal end distal to the guidewire proximal port 64. The mandrel is typically a metal member, such as a stainless steel or NiTi member, enhancing the pushability of the catheter 50. Alternatively, if the proximal section 61 comprises a hypotube, the distal end of the hypotube can include a skive as known to one of ordinary skill in the art and as described in U.S. Patent Publication No. 20012/0303054, the contents of which are fully incorporated herein by reference.

In the embodiment illustrated in FIGS. 6-8, as best shown in FIG. 7 showing an enlarged longitudinal cross sectional view of the catheter shaft taken along line 7-7 in FIG. 6, the distal section 62 of the outer tubular member 54 is a multilayered section with a first layer 67 and a second layer 68. The multilayered distal section 62 can be similar to the multilayered section of the catheter 10 discussed above in relation to the embodiment of FIGS. 1-3, and the discussion above relating to the first layer 27 and second layer 28 of the multilayered proximal section 25 of catheter 10 applies as well to first and second layers 67/68 of the multilayered distal section 62 of catheter 50. As such, the multilayer shaft section of FIG. 6 can include any of the features, materials, and or construction described above for the multilayer shaft section of the embodiments of FIGS. 1-3.

For example, the outer layer 68 can be any suitable polymer having a heat deflection temperature less than about 45° C. For example, the outer layer 68 can comprise a polymer selected from the group consisting of nylon 12, polyether block amide, and combinations thereof. Additionally or alternatively, outer layer 68 can comprise polyurethane. The second inner layer 67 can comprise a second polymer having a heat deflection temperature greater than about 53° C. For example, the inner layer 67 can comprise a polymer having a heat deflection temperature greater than about 53° C. selected from the group consisting of nylon 11, nylon 6, nylon 6,6, nylon 6,12, polyimide, polyamide-imide, polyetherimide, polypropylene, polyethylene terephthalate, polybutylene terephthalate, polyethereetherketone, and combinations thereof. As such a rapid exchange catheter that remains pushable even when exposed to body temperatures for an extended period of time is provided. Further, having a multilayer distal section 62 including an outer layer 68 of nylon or PEBAX can provide ease of bonding to the balloon 59, made of typical balloon materials such as nylon and/or PEBAX. For example, the proximal skirt section 70 of the balloon 59 can be fusion bonded to an outer layer 68 of distal shaft section 62, for example, by applying heat to the area of overlap.

In some embodiments, the distal section 62 of the outer tubular member 54 can include an intermediate layer 69, as best shown in FIG. 8. The intermediate layer can provide any of the benefits described above for intermediate layer 40 including but not limited to an improved moisture barrier, improved binding to the materials of the first layer and the second layer and reduced delamination during further processing of the catheter shaft (e.g., thermal bonding to other catheter components).

While the embodiment shown in FIG. 6 includes distal shaft section 62 that is multilayered, a proximal section 61 that is a single layer, and a midshaft section 63 that is single layer, any portion(s) of the catheter shaft can be multilayered to provide the catheter with the benefits described herein.

For example and in some embodiments, the proximal shaft section 61 and/or the midshaft section 63 of the outer tubular member 14 can comprise a multilayered tubular member and include at least an inner layer 67 and an outer layer 68 including any of the materials of construction, features, and/or layers as described herein.

In accordance with one embodiment of the disclosed subject matter, the balloon 19 or 59 can be a multilayer balloon (not shown). For example, the balloon can comprise a multilayered tubular member and include at least an inner layer 27 and an outer layer 28 including any of the materials of construction, features, and/or layers as described herein. For example, the multilayered balloon can be similar to the multilayered section of the catheter 10 discussed above in relation to the embodiment of FIGS. 1-3, and the discussion above relating to the first layer 27 and second layer 28 of the multilayered proximal section 25 of catheter 10 applies as well to first and second layers of multilayered balloon For example, the outer layer of the balloon can be any suitable polymer having a heat deflection temperature less than about 45° C. For example, the outer layer can comprise a polymer selected from the group consisting of nylon 12, polyether block amide, and combinations thereof. Additionally or alternatively, outer layer of the balloon can comprise polyurethane. The second inner layer of the balloon can comprise a second polymer having a heat deflection temperature greater than about 53° C. For example, the inner layer can comprise a polymer having a heat deflection temperature greater than about 53° C. selected from the group consisting of nylon 11, nylon 6, nylon 6,6, nylon 6,12, polyimide, polyamide-imide, polyetherimide, polypropylene, polyethylene terephthalate, polybutylene terephthalate, polyethereetherketone, and combinations thereof.

Alternatively, the balloon 19 or 59 can be composed of a wide variety of suitable materials, for example, nylon, co-polyamide such as Pebax (polyether block amide), polyester, co-polyester, polyurethane, polyethylene, or the like. More detailed lists of suitable materials are provided in U.S. Pat. Nos. 7,074,206, 7,828,766, and 8,052,638, each of which is hereby incorporated by reference in its entirety. In some embodiments, the first layer 27 can be made of a first polymer material having a first durometer, and the second layer 28 can be made of a second polymer material having a second durometer. As embodied herein, the second durometer can be greater than the first durometer, and the second layer can be an outer layer relative to the first layer. For example and not limitation, the balloon embodied herein has a first layer 27 composed of Pebax having a durometer of between about 55D and about 63D. The second layer 28 can be composed of, for example, Pebax having a durometer of between about 70D and about 72D Pebax.

Balloon 19 or 59 can have a noninflated configuration with wings wrapped around the balloon to form a low profile configuration for introduction and advancement within a patients body lumen. As a result, the balloon inflates to a nominal working diameter by unfolding and filling the molded volume of the balloon.

Balloon 19 can be formed with a working length 32 or 72, a distal cone section 34 or 74, and a distal skirt section 31 or 71. The distal skirt section 31 or 71 can have a first segment with a first diameter and a first wall thickness. The distal skirt section 31 or 71 can have a second segment with a second diameter and a second wall thickness. The second diameter can be greater than the first diameter and the second wall thickness is thinner than the first wall thickness as described in more detail in copending U.S. application Ser. No. 13/609,968, the contents of which is incorporated herein in its entirety.

For purpose of example and as embodied herein, the balloon 19 or 59 can be formed using a technique similar to that disclosed in U.S. Pat. Nos. 6,620,127, 7,828,766, 7,906,066 and 8,052,638, each of which is hereby incorporated by reference in its entirety. In some embodiments, the balloon 19 or 59 can be formed by melt-extruding a thermoplastic polymeric material to form a tube, then blow molding or forming in a mold into a blown balloon at a temperature less than an elevated temperature of the melt-extrusion under high pressure, for example between about 150 and about 500 psi. The blow molding can include placing the extruded tube within a mold or capture member. The extruded tube can be radially expanded under suitable conditions by introducing a pressurized fluid into the tube lumen until the outer surface of the extruded tube engages and conforms to the inner surface of the capture member. Furthermore, the polymeric material of the extruded tube can be biaxially oriented by axially expanding the extruded tube with a load applied on at least one end of the tube while radially stretching the extruded tube with a pressurized media in the tube lumen.

In accordance with another aspect, the balloon 19 or 59 can be formed using a two stage blow mold process such as disclosed in U.S. Patent Publication No. 2012/0065718, which is hereby incorporated by reference in its entirety.

For purpose of illustration and not limitation, and with reference to a coronary balloon catheter, the length of the balloon catheter disclosed herein can generally be about 108 to about 200 centimeters, preferably about 135 to about 150 centimeters, and typically about 145 centimeters for PTCA, and can have other suitable dimensions for other various applications. The outer tubular member can have, for purpose of example and not limitation, an outer diameter (OD) of about 0.042 inch (1.07 mm) to about 0.10 inch (2.54 mm), and an inner diameter (ID) of about 0.033 inch (0.84 mm) to about 0.088 inch (2.23 mm). The inner tubular member can have, for purpose of example and not limitation, an OD of about 0.022 inch (0.56 mm to about 0.050 inch (1.27 mm), and an ID of about 0.015 inch (0.38 mm) to about 0.040 inch (1.00 mm) depending on the diameter of the guidewire to be used with the catheter. For purpose of example and not limitation, the balloon can have a length of about 6 mm to about 100 mm, and an inflated working diameter of about 1.2 mm to about 100 mm.

When a catheter in accordance with the disclosed subject matter is used in an angioplasty procedure, the balloon catheter is advanced over the guidewire until the balloon is properly positioned across the stenosis. The balloon can be inflated in a conventional manner by introducing inflation fluid through the inflation lumen. After one or more inflations, the balloon is deflated and the catheter removed from the patient. A similar procedure is used when the balloon has a stent (not shown) mounted thereon for implanting the stent in the body lumen. For example, a radially expandable stent can be releasably mounted on the balloon 19 or 59 for delivery and deployment within the body lumen. The balloon catheter can be advanced in the body lumen with the balloon 19 or 59 in a noninflated configuration, and the balloon can be inflated by introducing inflation fluid into the balloon interior to expand the balloon 15 or 19 and stent mounted thereon. The balloon 19 or 59 can then be deflated to allow for repositioning or removal of the catheter from the body lumen, leaving the stent implanted in the body lumen

To the extent not previously discussed herein, the various catheter components can be formed and joined by conventional materials and methods. For example, one or more section of the tubular member can be a biaxially oriented tubular member and or can include a tapered region, as described in detail in U.S. Patent Publication No. 2013/0178795, which is incorporated by reference in its entirety. Likewise, inner tubular member can be formed by conventional techniques, such as disclosed in U.S. Pat. Nos. 6,277,093 and 6,217,547, each of which is incorporated by reference in its entirety. Additionally, although not illustrated, coiled or braided reinforcements can be included in the shaft at various locations, as is conventionally known as disclosed in U.S. Pat. No. 7,001,420, which is incorporated by reference in its entirety.

While the present disclosed subject matter has been described herein in terms of certain preferred embodiments, those skilled in the art will recognize that modifications and improvements can be made without departing from the scope of the disclosed subject matter. For example, although the catheters illustrated herein include balloon catheter, the catheter having at least one multilayered shaft section in accordance with the disclosed subject matter can be a variety of suitable catheters, including stent delivery catheters having a retractable sheath or sleeve over a working device (e.g., stent). In such embodiments, the sheath or sleeve can be a multilayer tubular member having any of the layers, materials of construction, features, and benefits described herein. While individual features of one embodiment of the disclosed subject matter may be discussed or shown in the drawings of the one embodiment and not in other embodiments, it should be apparent that individual features of one embodiment can be combined with one or more features of another embodiment or features from a plurality of embodiments.

1. An elongate, flexible catheter, comprising:
an elongated shaft having a proximal end, a distal end, and a lumen defined therein, the shaft including a tubular member having an outer layer comprising a first polymer selected from the group consisting of nylon 12, polyether block amide, and combinations thereof, and an inner layer comprising a second polymer having a heat deflection temperature greater than about 53° C. selected from the group consisting of nylon 11, nylon 6, nylon 6,6, nylon 6,12, polyamide-imide, polyetherimide, polypropylene, polyethylene terephthalate, polybutylene terephthalate, polyethereetherketone, and combinations thereof.

2. The elongate, flexible catheter of claim 1, wherein the inner layer and the outer layer are coextruded.

3. The elongate, flexible catheter of claim 1, wherein the second polymer is selected from the group consisting of nylon 11, nylon 6, nylon 6,6, nylon 6,12, polyamide-imide, polyetherimide, and combinations thereof, wherein the second polymer is directly bondable to the first polymer.

4. The elongate, flexible catheter of claim 1, wherein the second polymer is selected from the group consisting of polypropylene, polyethylene terephthalate, polybutylene terephthalate, polyethereetherketone, and combinations thereof, wherein the tubular member further includes an intermediate layer disposed between the first layer and the second layer.

5. The elongate, flexible catheter of claim 4, wherein the intermediate layer comprises a tie material directly bondable to the first and second polymers.

6. The elongate, flexible catheter of claim 5, wherein the tie material comprises ethylene acrylic acid copolymer, ethylene methacrylic acid copolymer, or combinations thereof.

7. The elongate, flexible catheter of claim 4, wherein the inner layer, the intermediate layer, and the outer layer are coextruded.

8. The elongate, flexible catheter of claim 1, further comprising a working device disposed proximate the distal end of the elongated shaft.

9. The elongate, flexible catheter of claim 1, wherein the working device comprises an inflatable balloon.

10. The elongate, flexible catheter of claim 1, wherein the first polymer comprises nylon 12.

11. The elongate, flexible catheter of claim 1, wherein the first polymer comprises PEBAX.

12. The elongate, flexible catheter of claim 1, wherein the tubular member forms one or more of a proximal shaft portion, a mid shaft portion, a distal shaft portion, or a sleeve disposed over a working device.

13. The elongate, flexible catheter of claim 1, wherein the catheter has improved pushability when exposed to body temperature as compared to a similar catheter having a shaft including a tubular member consisting of the first polymer.

14. A method of making an elongate, flexible catheter, comprising:
coextruding a tubular member having an outer layer and an inner layer to form at least a portion of an elongated shaft having a proximal end, a distal end, and a lumen defined therein, the outer layer comprising a first polymer selected from the group consisting of nylon 12, polyether block amide, and combinations thereof, and the inner layer comprising a second polymer having a heat deflection temperature greater than about 53° C.
disposing a working device proximate the distal section of the elongated shaft.

15. The method of claim 14, wherein the second polymer is selected from the group consisting of nylon 11, nylon 6, nylon 6,6, nylon 6,12, polyimide, polyimide-imide, polyetherimide, and combinations thereof, wherein the second polymer is directly bondable to the first polymer.

16. The method of claim 14, wherein the second polymer is selected from the group consisting of polypropylene, polyethylene terephthalate, polybutylene terephthalate, polyethereetherketone, and combinations thereof, wherein the tubular member further comprises an intermediate layer disposed between the first layer and the second layer.

17. The method of claim 16, wherein the intermediate layer comprises a tie material directly bondable to the first and second polymers.

18. The method of claim 17, wherein the tie material comprises ethylene acrylic acid copolymer, ethylene methacrylic acid copolymer, or combinations thereof.

19. The method of claim 14, wherein the first polymer comprises nylon 12.

20. The method of claim 14, wherein the first polymer comprises PEBAX.

21. The method of claim 14, wherein the portion of the elongated shaft comprises one or more of a proximal shaft portion, a mid shaft portion, a distal shaft portion, or a sleeve disposed over the working device.

22. The method of claim 14, wherein the catheter has improved pushability when exposed to body temperature as compared to a similar catheter having a shaft including a tubular member consisting of the first polymer.
